(19) United States
(12) Patent Application Publication
(10) Pub. No.: US 2025/0387107 A1
(43) Pub. Date: Dec. 25, 2025

(54) ENDOSCOPIC DEVICE AND TREATMENT TOOL INSERTION METHOD

(71) Applicant: OLYMPUS CORPORATION, Tokyo (JP)

(72) Inventor: Kazuhiro KAN, Kunitachi-shi (JP)

(73) Assignee: OLYMPUS CORPORATION, Tokyo (JP)

(21) Appl. No.: 19/036,162

(22) Filed: Jan. 24, 2025

Related U.S. Application Data
(60) Provisional application No. 63/663,406, filed on Jun. 24, 2024.

(51) Int. Cl. A61B 17/00; A61F 2/82; A61F 2/95

(52) U.S. Cl. CPC A61B 17/00234; A61B 2017/00296; A61B 2017/00561; A61F 2/82; A61F 2/95

(57) ABSTRACT

What is provided is an endoscopic device including: a sheath extending in a longitudinal direction thereof; a treatment tool opening portion that communicates with a treatment tool lumen inserted through the sheath in the longitudinal direction and allows a treatment tool inserted through the treatment tool lumen to project to and retract from a distal end side of the sheath in the longitudinal direction; and a suction opening portion communicating with a suction lumen inserted through the sheath in the longitudinal direction, wherein the treatment tool opening portion and the suction opening portion are provided in an insertion region that is disposed inside bile and pancreatic ducts when the sheath is inserted into the bile and pancreatic ducts, and the suction opening portion is able to suck an inner wall of the bile and pancreatic ducts.

### BACKGROUND OF THE INVENTION

### Field of the Invention

The present invention relates to an endoscopic device and a treatment tool insertion method. Priority is claimed on U.S. Provisional Application No. 63/663,406, filed Jun. 24, 2024, the content of which is incorporated herein by reference.

### Description of Related Art

In the related art, there is a treatment tool such as a catheter or the like inserted into bile and pancreatic ducts for the purpose of examination and treatment of the bile and pancreatic ducts (for example, Patent Document 1). For example, an examination accompanied by a procedure of inserting a treatment tool into bile and pancreatic ducts includes endoscopic retrograde cholangiopancreatography (ERCP).

### Patent Documents

[Patent Document 1] Japanese Unexamined Patent Application, First Publication No. 2023-135253

### SUMMARY OF THE INVENTION

However, since bile and pancreatic ducts have narrowed and meandering portions, it is difficult to insert a treatment tool into the bile and pancreatic ducts, and there is a need to improve ease of insertion of the treatment tool.

In light of the above circumstances, an object of one aspect of the present invention is to provide an endoscopic device and a treatment tool insertion method in which ease of insertion is improved when a treatment tool is inserted into bile and pancreatic ducts.

In order to solve the above problem, one aspect of the present invention proposes the following means.

An endoscopic device according to one aspect of the present invention includes: a sheath extending in a longitudinal direction thereof; a treatment tool opening portion that communicates with a treatment tool lumen inserted through the sheath in the longitudinal direction and allows a treatment tool inserted through the treatment tool lumen to project to and retract from a distal end side of the sheath in the longitudinal direction; and a suction opening portion communicating with a suction lumen inserted through the sheath in the longitudinal direction, wherein the treatment tool opening portion and the suction opening portion are provided in an insertion region that is disposed inside bile and pancreatic ducts when the sheath is inserted into the bile and pancreatic ducts, and the suction opening portion is able to suck an inner wall of the bile and pancreatic ducts.

A treatment tool insertion method according to one aspect of the present invention includes: a sheath insertion step of inserting a distal end portion of a sheath extending in a longitudinal direction thereof into an inside of bile and pancreatic ducts through an opening portion of a papilla; a suction step of sucking an inner wall of the bile and pancreatic ducts using a suction opening portion provided in an insertion region of the sheath inserted into the inside of the bile and pancreatic ducts; a traction step of performing traction of the sheath, which has sucked the inner wall of the bile and pancreatic ducts, toward a proximal end side in the longitudinal direction, thereby causing the sheath to perform traction of the papilla; and a treatment tool insertion step of advancing a treatment tool through a treatment tool opening portion of the sheath, which has performed traction of the papilla, and inserting the treatment tool into the inside of the bile and pancreatic ducts.

According to the endoscopic device and the treatment tool insertion method of one aspect of the present invention, the endoscopic device and the treatment tool insertion method in which ease of insertion is improved when the treatment tool is inserted into bile and pancreatic ducts can be provided.

### BRIEF DESCRIPTION OF THE DRAWINGS

FIG. 1 is an overall view of an endoscope system according to the present embodiment.

FIG. 2 is an overall view showing an endoscopic device of the endoscope system.

FIG. 3 is a perspective view of a distal end portion of the endoscopic device.

FIG. 4 is a cross-sectional view along line IV-IV in FIG. 3.

FIG. 5 is a diagram showing bile and pancreatic ducts serving as a treatment target.

FIG. 6 is a diagram showing a suction step of a treatment tool insertion method according to the present embodiment.

FIG. 7 is a diagram showing a guide wire insertion step of the treatment tool insertion method.

FIG. 8 is a diagram showing the guide wire insertion step of the treatment tool insertion method.

FIG. 9 is a diagram showing a guide wire placement step of the treatment tool insertion method.

FIG. 10 is a diagram showing a stent insertion step of the treatment tool insertion method.

FIG. 11 is a perspective view showing a modified example of a treatment unit of the endoscopic device.

FIG. 12 is a cross-sectional view along line XII-XII in FIG. 11.

FIG. 13 is a perspective view showing another modified example of the treatment unit.

FIG. 14 is a cross-sectional view along line XIV-XIV in FIG. 12.

FIG. 15 is an overall view showing a modified example of the endoscopic device.

FIG. 16 is a perspective view showing another modified example of the treatment unit.

FIG. 17 is a cross-sectional view along line XVII-XVII in FIG. 16.

### DETAILED DESCRIPTION OF THE INVENTION

An endoscope system 300 according to one embodiment of the present invention will be described with reference to the drawings.

### Endoscope System 300

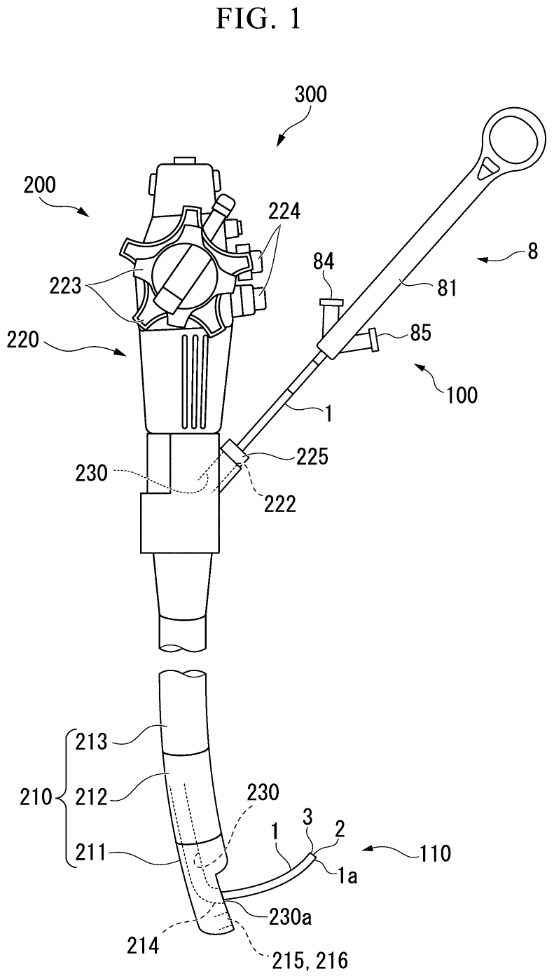

FIG. 1 is an overall view of the endoscope system 300 according to the present embodiment.

The endoscope system 300 includes an endoscopic device 100 and an endoscope 200. The endoscopic device 100 is inserted into the endoscope 200 for use.

### Endoscope 200

The endoscope 200 is a known side-viewing type flexible endoscope, and includes an elongated insertion unit 210 and an endoscope operation unit 220 provided at a proximal end portion of the insertion unit 210. Also, the endoscope 200 may be a direct-viewing type flexible endoscope.

The insertion unit 210 includes a distal end rigid portion 211 provided at its distal end portion, a bending portion 212 that is provided on a proximal end side of the distal end rigid portion 211 and can be operated to be bent, and a flexible tube portion 213 provided on a proximal end side of the bending portion 212.

A light guide 215 and an imaging unit 216 having a CCD are provided on a side surface of the distal end rigid portion 211 in a state in which they are exposed to the outside.

A treatment tool channel 230 for insertion of the endoscopic device 100 is formed in the insertion unit 210.

A distal end opening portion 230a of the treatment tool channel 230 opens on the side surface of the distal end rigid portion 211. A proximal end portion of the treatment tool channel 230 extends to the endoscope operation unit 220.

A raising base 214 is provided in the distal end rigid portion 211 of the treatment tool channel 230. A proximal end portion of the raising base 214 is rotatably supported by the distal end rigid portion 211.

A raising base operation wire (not shown) fixed to a distal end portion of the raising base 214 extends through the insertion unit 210 to the endoscope operation unit 220.

The bending portion 212 is configured to be freely bent in vertical and horizontal directions. A distal end portion of a bending operation wire (not shown) is fixed to a distal end side of the bending portion 212. The bending operation wire extends through the insertion unit 210 to the endoscope operation unit 220.

The endoscope operation unit 220 is provided with a knob 223 for operating the bending operation wire and a switch 224 for operating the imaging unit 216, and the like. A surgeon can bend the bending portion 212 in a desired direction by operating the knob 223.

Also, the endoscope operation unit 220 may be provided with a knob or the like for operating the raising base operation wire. The surgeon can rotate the raising base 214 in any direction and at any angle by operating the endoscope operation unit 220.

A forceps port 222 that communicates with the treatment tool channel 230 is provided on a distal end side of the endoscope operation unit 220. The surgeon can insert the endoscopic device 100 from the forceps port 222. A forceps plug 225 is attached to the forceps port 222 to prevent leakage of bodily fluids.

### Endoscope Device 100

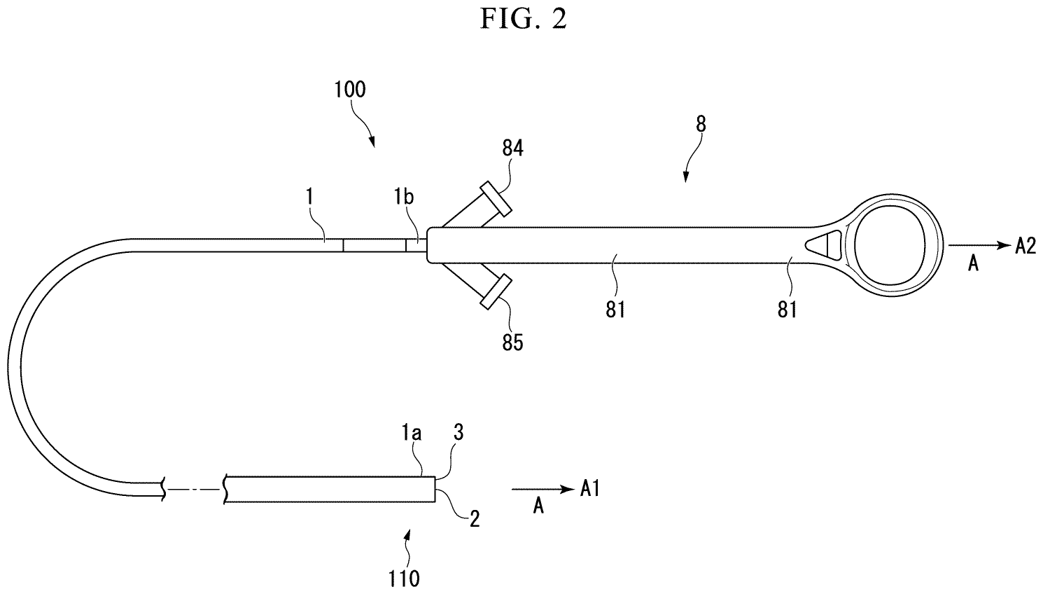

FIG. 2 is an overall view showing the endoscopic device 100.

The endoscopic device 100 includes a sheath 1 and an operation unit 8.

In the following description, in a longitudinal direction A of the endoscopic device 100, a side inserted into a patient's body will be referred to as a “distal end side (distal side) A1,” and a side of the operation unit 8 will be referred to as a “proximal end side (proximal side) A2.”

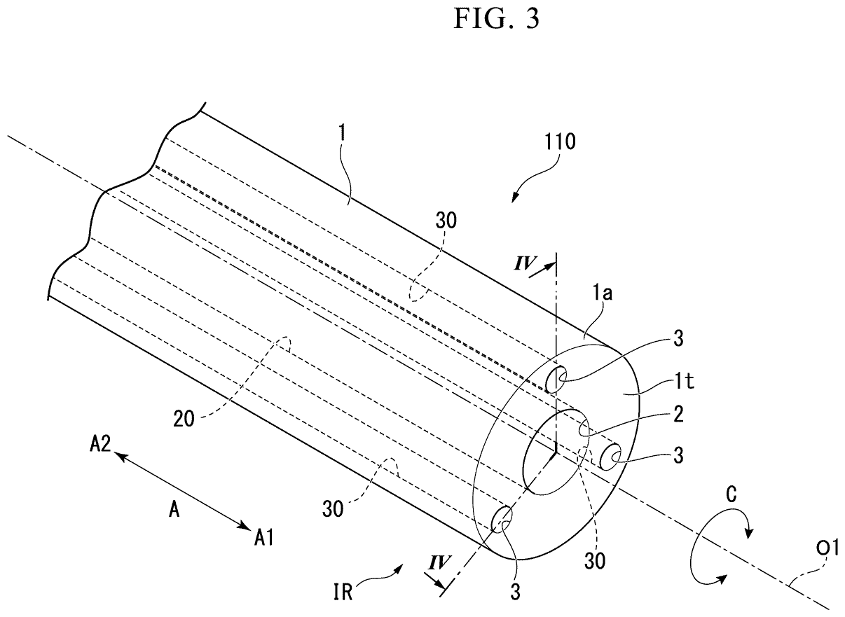

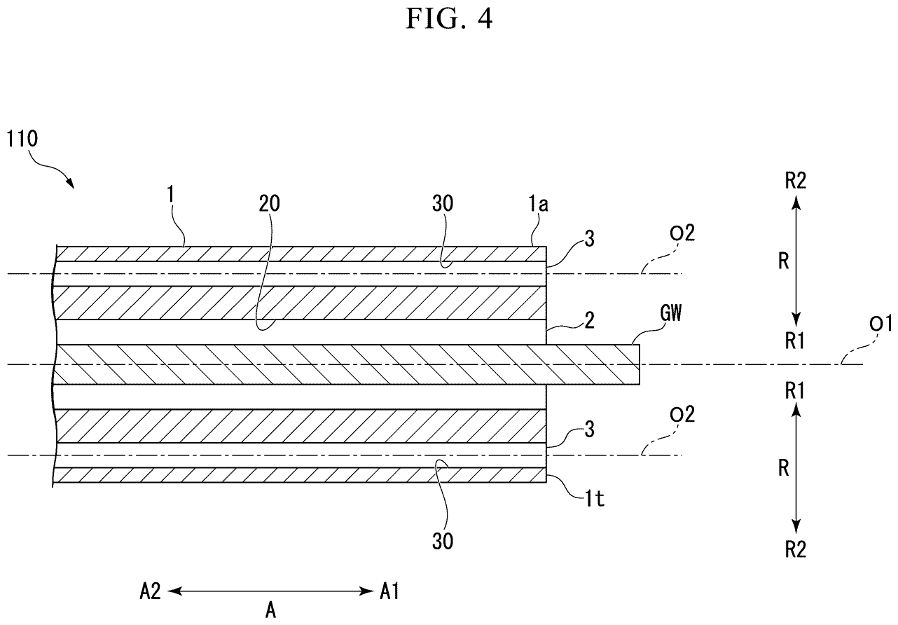

FIG. 3 is a perspective view of a distal end portion of the endoscopic device 100. FIG. 4 is a cross-sectional view along line IV-IV in FIG. 3.

The distal end portion of the endoscopic device 100 is provided with a treatment tool opening portion 2 and a suction opening portion 3.

In the following description, the distal end portion of the endoscopic device 100 provided with the treatment tool opening portion 2 and the suction opening portion 3 will be referred to as a “treatment unit 110.”

The sheath 1 is an elongated member that has flexibility and extends from a distal end 1a to a proximal end 1b thereof. The sheath 1 has an outer diameter that allows it to be inserted into the treatment tool channel 230 of the endoscope 200.

As shown in FIG. 1, in a state in which the sheath 1 is inserted into the treatment tool channel 230, the distal end 1a of the sheath 1 can project and retract from the distal end opening portion 230a of the treatment tool channel 230. The sheath 1 may be insulating.

As shown in FIG. 3, a treatment tool lumen 20 and a suction lumen 30 are inserted through the sheath 1 in the longitudinal direction A.

The treatment tool opening portion 2 is an opening portion that communicates with the treatment tool lumen 20 and is provided on a distal end surface 1t of the sheath 1. Here, the distal end surface 1t of the sheath 1 is a surface oriented toward a distal end side A1 in the treatment unit 110.

In the present embodiment, the distal end surface 1t of the sheath 1 is a flat surface extending perpendicular to the longitudinal direction A, but the distal end surface 1t of the sheath 1 may have an uneven shape.

The suction opening portion 3 is an opening portion that communicates with the suction lumen 30 and is provided on the distal end surface 1t of the sheath 1.

In the following description, as shown in FIG. 4, in a radial direction R of the sheath 1, a side of the sheath 1 coming closer to a central axis O1 will be referred to as an “inner side R1,” and a side away from the central axis O1 will be referred to as an “outer side R2.”

In the present embodiment, the suction opening portion 3 is provided on the outer side R2 in the radial direction R from the treatment tool opening portion 2. In addition, the suction lumen 30 is provided on the outer side R2 from the treatment tool lumen 20.

The suction opening portion 3 does not have to be disposed on the outer side R2 from the treatment tool opening portion 2, and the suction opening portion 3 and the treatment tool opening portion 2 may be disposed at, for example, substantially equal positions in the radial direction R.

In the present embodiment, three suction opening portions 3 are provided on the distal end surface 1t of the sheath 1. In addition, three suction lumens 30 that respectively communicate with the three suction opening portions 3 are formed in the sheath 1.

The number of suction opening portions 3 provided in the treatment unit 110 may be less than three, or may be four or more.

In the present embodiment, a central axis of the treatment tool lumen 20 coincides with the central axis O1 of the sheath 1. In addition, central axes O2 of the suction lumens 30 extend in the longitudinal direction A parallel to the central axis O1 of the sheath 1.

The central axis of the treatment tool lumen 20 does not have to coincide with the central axis O1 of the sheath 1. Also, the central axes O2 of the suction lumens 30 do not have to be parallel to the central axis O1 of the sheath 1.

In the present embodiment, the three suction opening portions 3 are disposed at equal intervals in a circumferential direction C of the central axis O1 of the sheath 1, as shown in FIG. 3.

The three suction opening portions 3 do not have to be strictly disposed at equal intervals in the circumferential direction C.

The treatment tool lumen 20 has a size that allows a predetermined treatment tool to be inserted therethrough. A guide wire GW shown in FIG. 4 is an example of the treatment tool that is inserted through the treatment tool lumen 20. The guide wire GW can project and retract from the treatment tool opening portion 2. The guide wire GW is an elongated guide wire that extends in the longitudinal direction A.

The treatment tool that projects and retracts from the treatment tool opening portion 2 is not limited to the guide wire GW, and may be various treatment tools used in procedures using the endoscope system 300.

In the treatment unit 110, the treatment tool opening portion 2 and the suction opening portions 3 are provided in an insertion region IR. The insertion region IR is a region that is disposed inside bile and pancreatic ducts when the sheath 1 is inserted into the bile and pancreatic ducts in a treatment tool insertion method, which will be described later.

As shown in FIG. 3, the insertion region IR is a region in which a distal end portion of the treatment unit 110 is included.

The operation unit 8 is provided on the proximal end side A2 of the sheath 1. The operation unit 8 includes an operation unit main body 81, a treatment tool port 84, and a suction port 85.

A distal end portion of the operation unit main body 81 is connected to the proximal end 1b of the sheath 1.

The treatment tool port 84 is an opening through which a treatment tool such as the guide wire GW can be inserted into the treatment tool lumen 20. The treatment tool such as the guide wire GW inserted from the treatment tool port 84 passes through the treatment tool lumen 20 and protrudes from the treatment tool opening portion 2 to the distal end side A1.

The suction port 85 is an opening that communicates with the suction lumens 30. For example, a pump device can be connected to the suction port 85 and the suction lumens 30 can be brought to a negative pressure by driving the pump device.

### Operation of Endoscope System 300

Next, an operation of the endoscope system 300 according to the present embodiment will be described. Specifically, a procedure of placing a stent inside the bile and pancreatic ducts using endoscopic retrograde cholangiopancreatography (ERCP) will be described.

The operation of the endoscope system 300 described below is an example of a treatment tool insertion method using the endoscope system 300.

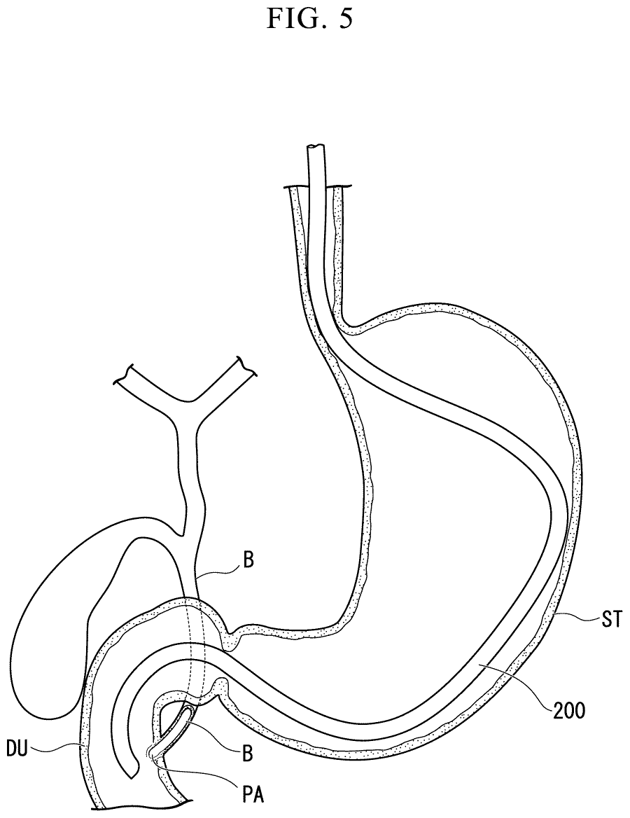

FIG. 5 is a diagram showing bile and pancreatic ducts B serving as a treatment target. In the present embodiment, the bile and pancreatic ducts B include a bile duct, a pancreatic duct, and a portion at which the bile duct and the pancreatic duct join to form a single duct path.

### Endoscope Insertion Step

First, the surgeon performs an endoscope insertion step. In the endoscope insertion step, the surgeon inserts the insertion unit 210 of the endoscope 200 into the patient's lumen through a natural opening such as the mouth. If necessary, the surgeon operates the endoscope operation unit 220 to bend the bending portion 212.

The surgeon passes the distal end rigid portion 211 of the endoscope 200 through the stomach ST and inserts it into the duodenum DU. In this case, the surgeon disposes the distal end rigid portion 211 in the vicinity of the papilla PA so that a direction in which the endoscopic device 100 protrudes from the endoscope 200 coincides with a direction in which the bile and pancreatic ducts extend. The surgeon may insert the insertion unit 210 of the endoscope 200 into the patient's lumen using an overtube.

### Sheath Insertion Step

Next, the surgeon performs a sheath insertion step.

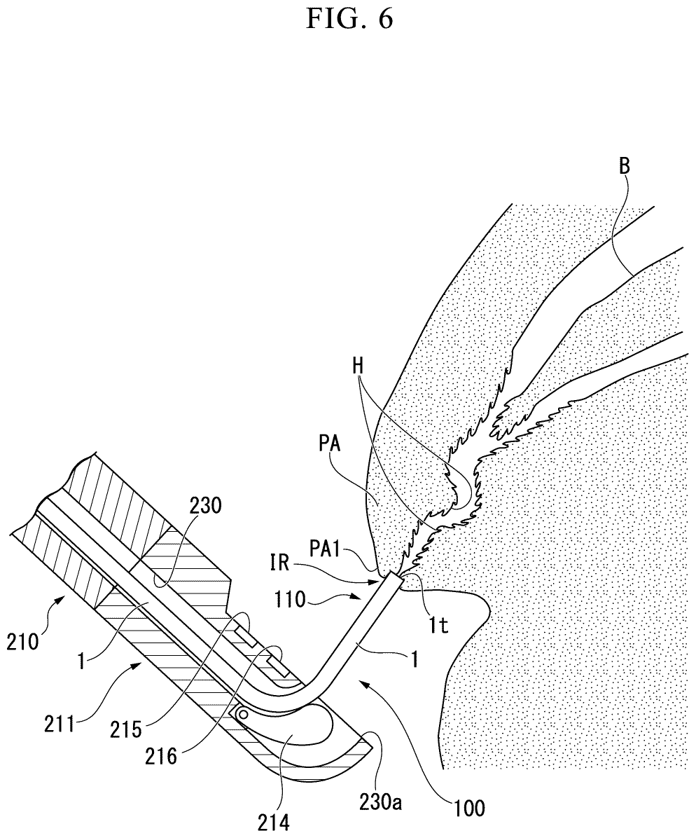

FIG. 6 is a diagram showing a suction step of the treatment tool insertion method according to the present embodiment. The suction step will be described later.

In the sheath insertion step, the surgeon inserts the endoscopic device 100 into the bile and pancreatic ducts B from an opening portion PA1 of the papilla PA.

Specifically, the endoscopic device 100 is inserted into the treatment tool channel 230 of the endoscope 200 to cause the treatment unit 110 of the endoscopic device 100 to protrude from the distal end opening portion 230a, and the distal end portion of the sheath 1 is inserted into an inside of the bile and pancreatic ducts B from the opening portion PA1 of the papilla PA.

In this case, since at least the insertion region IR of the treatment unit 110 is inserted into the inside of the bile and pancreatic ducts B, the treatment tool opening portion 2 and the suction opening portions 3 are disposed inside the bile and pancreatic ducts B.

### Suction Step

Next, the surgeon performs the suction step. In the suction step, the surgeon sucks an inner wall of the bile and pancreatic ducts B using the suction opening portions 3 of the treatment unit 110.

In this case, the surgeon drives the pump device connected to the suction port 85 to create a negative pressure in the suction lumens 30. By creating the negative pressure in the suction lumens 30, the surgeon sucks the inner wall of the bile and pancreatic ducts B using the suction opening portions 3 to cause the inner wall of the bile and pancreatic ducts B to be adhered to the distal end surface 1t of the sheath 1.

In the present embodiment, the inner wall of the bile and pancreatic ducts B sucked by the suction opening portions 3 is a portion of the bile and pancreatic ducts B that is closer to the opening portion PA1 than the portion at which the bile duct and pancreatic duct join.

For example, as shown in FIG. 6, the suction opening portions 3 suck a duct wall inside the papilla PA in a region closer to the opening portion PA1 of the papilla PA than a portion of the bile and pancreatic ducts B at which pleats H are present and cause it to be adhered.

### Traction Step

Next, the surgeon performs a traction step.

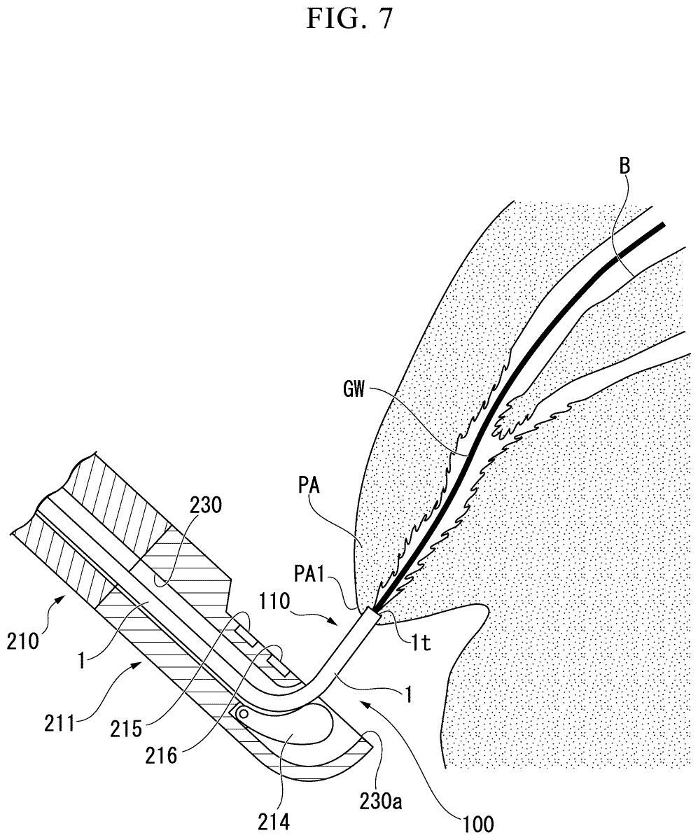

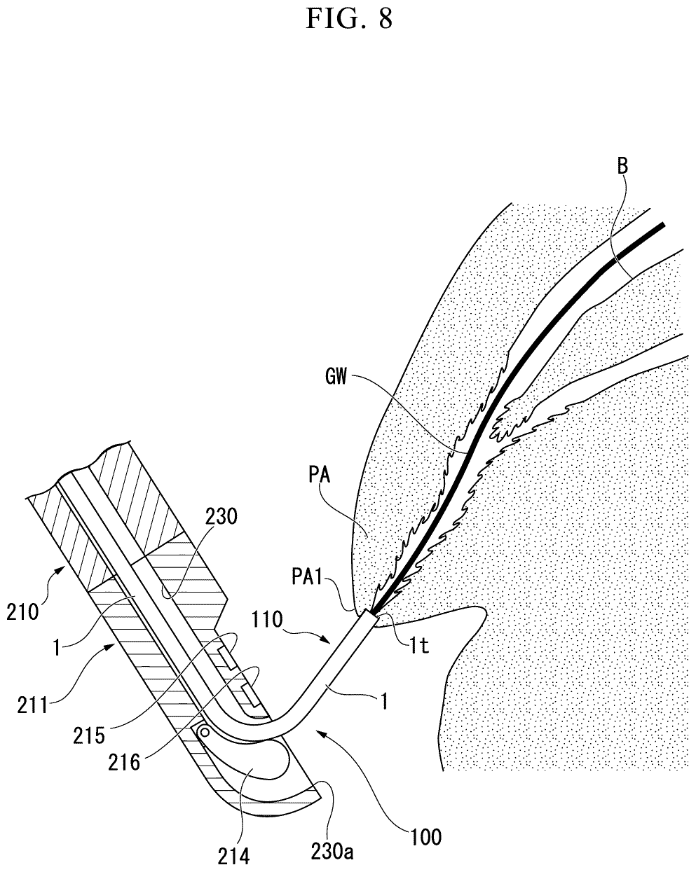

FIGS. 7 and 8 are diagrams showing a guide wire insertion step of the treatment tool insertion method. The guide wire insertion step will be described later.

In the traction step, the surgeon performs traction of the sheath 1, which has sucked the inner wall of the bile and pancreatic ducts B, toward the proximal end side A2, thereby performing traction of the papilla PA to the sheath 1.

In this case, as shown in FIG. 7, the surgeon may move the endoscopic device 100 toward the proximal end side A2 relative to the endoscope 200 to cause the sheath 1 to perform traction of the papilla PA.

Also, as shown in FIG. 8, the surgeon may perform an operation (angle operation) of bending the bending portion 212 so that the distal end rigid portion 211 of the endoscope 200 moves away from the papilla PA, thereby causing the sheath 1 to perform traction of the papilla PA.

### Guide Wire Insertion Step

Next, the surgeon performs the guide wire insertion step. In the guide wire insertion step, the surgeon advances the guide wire GW inserted through the treatment tool lumen 20 to cause the guide wire GW to protrude from the treatment tool opening portion 2. The surgeon further advances the guide wire GW protruding from the treatment tool opening portion 2 to cause the guide wire GW to be inserted into the bile and pancreatic ducts B.

When the guide wire GW is inserted into the bile and pancreatic ducts B, the bile and pancreatic ducts B may have a shape that makes it difficult to insert the guide wire GW. For example, when the bile and pancreatic ducts B are sharply bent or meandering at the papilla PA, or when a part of the bile and pancreatic ducts B is narrowed due to the pleats H or the like, the guide wire GW is less likely to be inserted into the bile and pancreatic ducts B.

In addition, the bile and pancreatic ducts B in the vicinity of the pleats H and the opening portion PA1 may be very soft, and the bile and pancreatic ducts B may be easily bent or buckled simply by lightly pressing the vicinity of the opening portion PA1 with the endoscopic device 100.

In particular, when the bile and pancreatic ducts B has a narrow distal segment (NDS; a portion in which the duodenal penetration portion distal to the extrahepatic bile duct is narrowed due to the influence of the sphincter of Oddi), it becomes difficult to insert the guide wire GW into the bile and pancreatic ducts B.

In the guide wire insertion step of the present embodiment, by causing the sheath 1 to perform traction of the papilla PA to stretch the papilla PA, the shape of the bile and pancreatic ducts B can be made to a shape that makes it easier to insert the guide wire GW.

It is desirable for the surgeon to perform traction of the papilla PA with the sheath 1 until the shape of the bile and pancreatic ducts B becomes straight. Also, the surgeon does not necessarily have to perform traction of the papilla PA until the shape of the bile and pancreatic ducts B becomes straight. The surgeon may pull the papilla PA until the shape of the bile and pancreatic ducts B changes to one that makes it easier to insert the guide wire GW. For example, the surgeon may perform traction of the papilla PA until the shape of the bile and pancreatic ducts B changes to a bent shape that is gently bent.

In addition, in the treatment tool insertion method of the present embodiment, the insertion region IR of the treatment unit 110 is inserted into the bile and pancreatic ducts B in the sheath insertion step described above, and the inner wall of the bile and pancreatic ducts B is sucked and adhered to the suction opening portions 3 in the suction step described above.

For that reason, as compared to when the opening portion PA1 is sucked and adhered from the outside of the papilla PA, deformation of the opening portion PA1 can be inhibited, and an insertion path large enough for inserting the guide wire GW can be secured.

Also, since the guide wire GW is caused to protrude from the treatment tool opening portion 2 provided at the distal end portion of the sheath 1 inserted into the bile and pancreatic ducts B, the guide wire GW can be reliably inserted into the bile and pancreatic ducts B.

Further, since the suction opening portions 3 sucking the inner wall of the bile and pancreatic ducts B are provided on the outer side R2 from the treatment tool opening portion 2 through which the guide wire GW projects and retracts, clogging of the treatment tool opening portion 2 caused by the inner wall of the bile and pancreatic ducts B can be inhibited, and the guide wire GW can be reliably inserted into the bile and pancreatic ducts B.

As described above, the guide wire GW is an example of a treatment tool used in a procedure using the endoscope system 300. In the following description, the guide wire insertion step is also referred to as a treatment tool insertion step.

### Guide Wire Placement Step

Next, the surgeon performs a guide wire placement step.

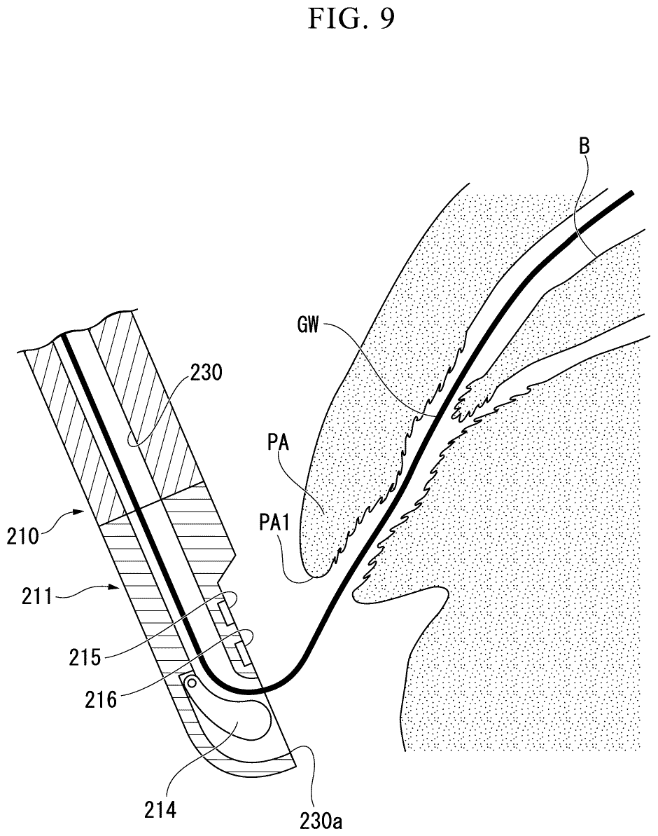

FIG. 9 is a diagram showing the guide wire placement step of the treatment tool insertion method. In the guide wire placement step, the surgeon removes the endoscopic device 100 from the bile and pancreatic ducts B while leaving the guide wire GW in place inside the bile and pancreatic ducts B. Thus, only the guide wire GW is placed in the bile and pancreatic ducts B.

### Stent Placement Step

Next, the surgeon performs a stent placement step.

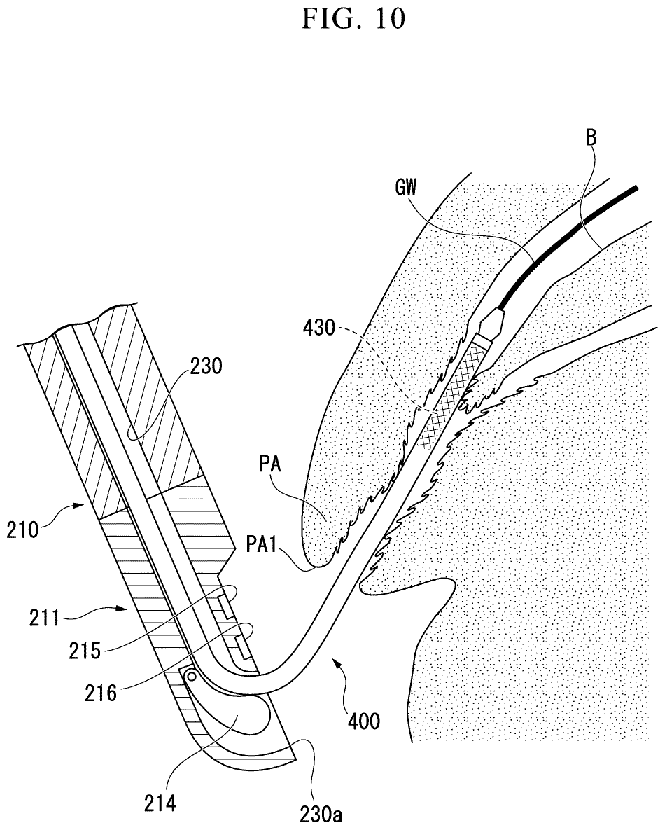

FIG. 10 is a diagram showing the stent placement step of the treatment tool insertion method. In the stent placement step, the surgeon inserts a stent delivery device 400 along the guide wire GW into the bile and pancreatic ducts B.

The surgeon operates the stent delivery device 400 to move the stent delivery device 400 along the guide wire GW toward the liver side, and moves a portion of the stent delivery device 400 in which a stent 430 is housed to the vicinity of a stenosis site.

The surgeon operates the stent delivery device 400 to expose the stent 430 housed in the stent delivery device 400, and places the stent 430 in the bile and pancreatic ducts B.

Although the procedure for placing the stent 430 has been shown as an example of the operation of the endoscope system 300, treatments using the endoscope system 300 are not limited to the procedure for placing the stent 430. For example, the endoscope system 300 can also be used for procedures using a stone retrieval or stone smash basket or a stone retrieval balloon using the placed guide wire GW.

The endoscopic device 100 according to the present embodiment includes the sheath 1 extending in the longitudinal direction A, the treatment tool opening portion 2 that communicates with the treatment tool lumen 20 inserted through the sheath 1 in the longitudinal direction A and allows the treatment tool (for example, the guide wire GW) inserted through the treatment tool lumen 20 to project and retract from the distal end side A1 of the sheath 1, and the suction opening portions 3 that communicate with the suction lumens 30 inserted through the sheath 1 in the longitudinal direction A.

The treatment tool opening portion 2 and the suction opening portions 3 are provided in the insertion region IR that is disposed inside the bile and pancreatic ducts B when the sheath 1 is inserted into the bile and pancreatic ducts B. The suction opening portions 3 are capable of sucking the inner wall of the bile and pancreatic ducts B.

The treatment tool insertion method according to the present embodiment includes the sheath insertion step of inserting the distal end portion of the sheath 1 into the bile and pancreatic ducts B from the opening portion PA1 of the papilla PA, the suction step of sucking the inner wall of the bile and pancreatic ducts B using the suction opening portions 3 provided in the insertion region IR of the sheath 1 inserted into the bile and pancreatic ducts B, the traction step of performing traction of the sheath 1, which has sucked the inner wall of the bile and pancreatic ducts B, toward the proximal end side A2 to cause the sheath 1 to perform traction of the papilla PA, and the treatment tool insertion step of advancing the treatment tool (for example, the guide wire GW) from the treatment tool opening portion 2 of the sheath 1, which has performed traction of the papilla PA, to insert the treatment tool into the bile and pancreatic ducts B.

According to the endoscopic device 100 and the treatment tool insertion method of the present embodiment, it is possible to provide the endoscopic device 100 and the treatment tool insertion method in which ease of insertion is improved when a treatment tool is inserted into the bile and pancreatic ducts B.

Although one embodiment of the present invention has been described above in detail with reference to the drawings, specific configurations are not limited to the present embodiment, and changes in design in the range without departing from the gist of the present invention are also included. In addition, constituent elements shown in the above-described embodiment and modified examples can be appropriately combined to configure the present invention.

### Modified Example 1

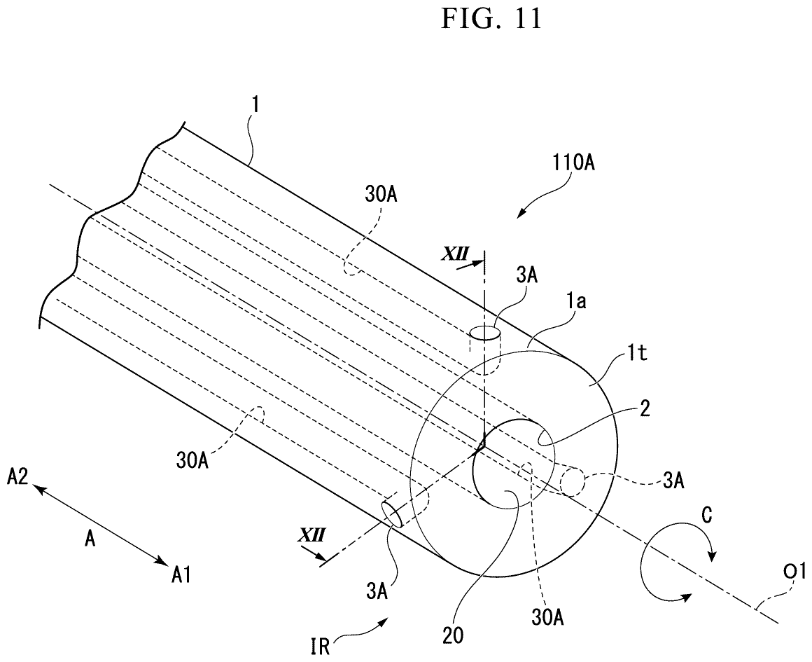

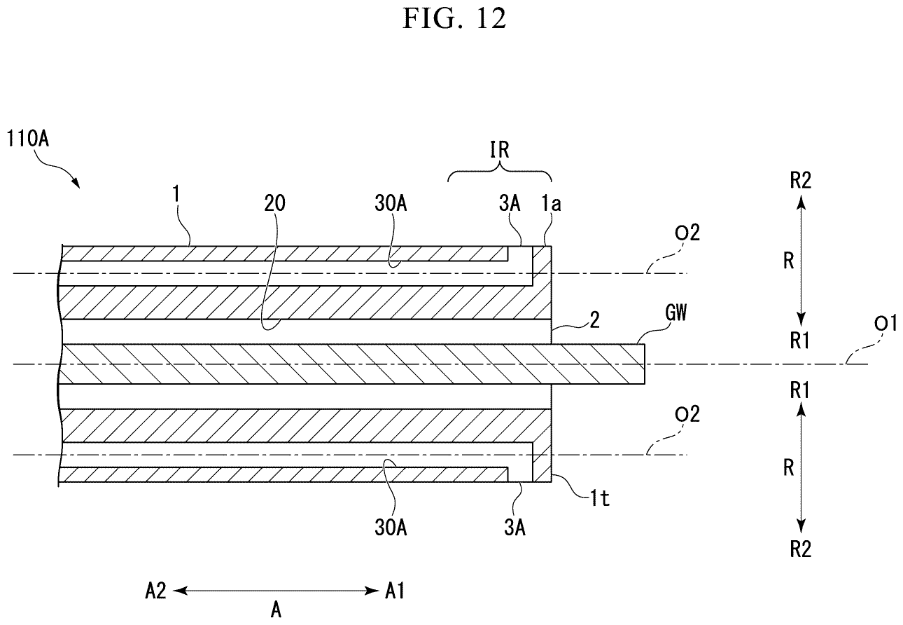

FIG. 11 is a perspective view showing a treatment unit 110A, which is a modified example of the treatment unit 110. FIG. 12 is a cross-sectional view along line XII-XII in FIG. 11.

The treatment unit 110A is provided with the treatment tool opening portion 2 provided on the distal end surface 1t of the sheath 1, and suction opening portions 3A provided on an outer circumferential surface of the sheath 1.

In the present modified example, the treatment unit 110A is provided with three suction opening portions 3A. Also, the three suction opening portions 3A are disposed at equal intervals in the circumferential direction C of the central axis O1 of the sheath 1. The three suction opening portions 3A do not have to be strictly disposed at equal intervals in the circumferential direction C.

As shown in FIG. 12, the suction opening portions 3A are provided in the insertion region IR. The suction opening portions 3A open toward the outer side R2, and thus, when the treatment unit 110A is inserted into the bile and pancreatic ducts B in the sheath insertion step and the inner wall of the bile and pancreatic ducts B is sucked by the suction opening portions 3A in the suction step, the inner wall of the bile and pancreatic ducts B can be stably sucked and adhered.

### Modified Example 2

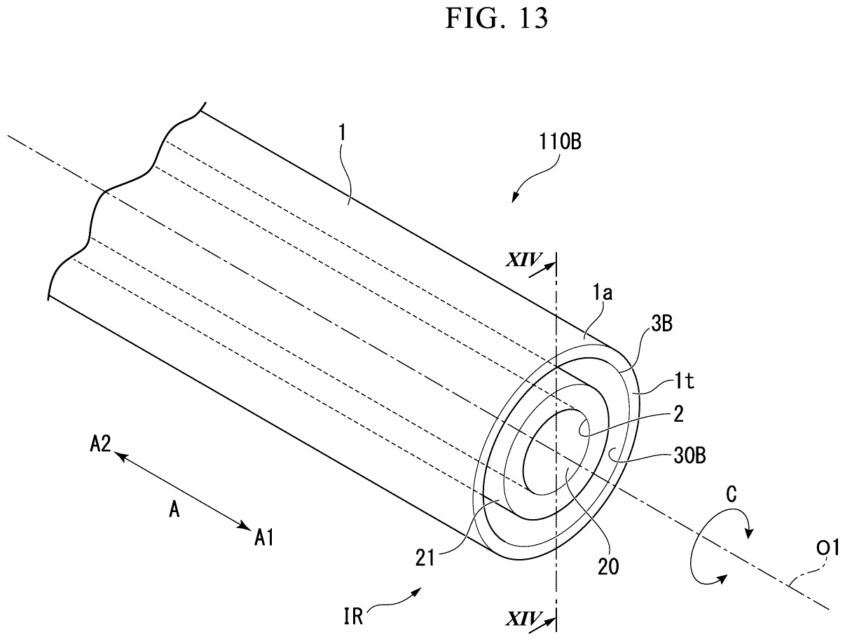

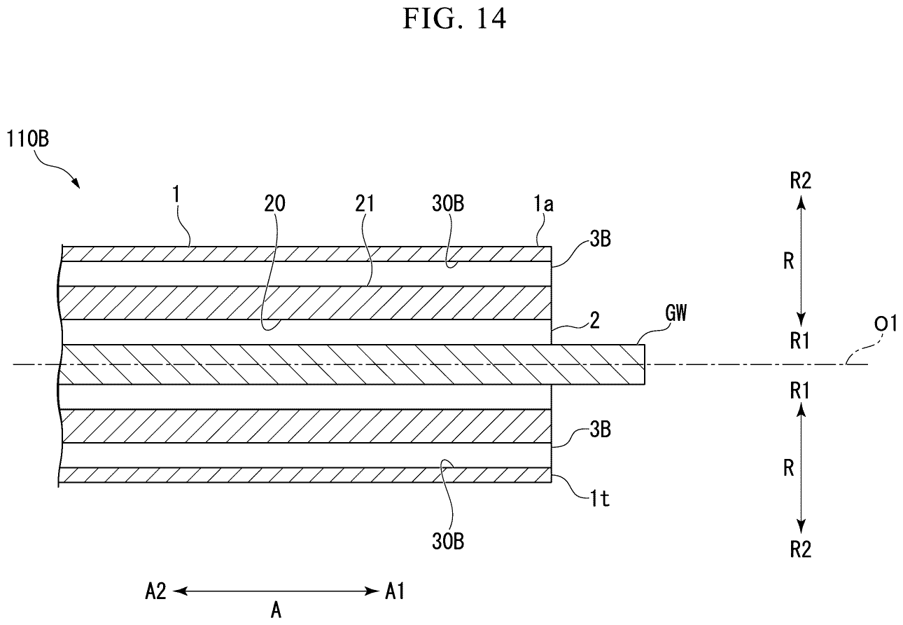

FIG. 13 is a perspective view showing a treatment unit 110B, which is a modified example of the treatment unit 110. FIG. 14 is a cross-sectional view along line XIV-XIV in FIG. 13.

In the treatment unit 110B, a central axis of a suction lumen 30B, which communicates with a suction opening portion 3B provided on the distal end surface 1t of the sheath 1, coincides with the central axis O1 of the sheath 1. The central axis of the suction lumen 30B does not have to strictly coincide with the central axis O1.

In addition, the treatment tool opening portion 2 is provided in an inner sheath 21 that is inserted through the suction lumen 30B in the longitudinal direction A. The treatment tool lumen 20 that communicates with the treatment tool opening portion 2 is inserted through the inner sheath 21 in the longitudinal direction A.

The inner sheath 21 extends, for example, from the distal end 1a to the proximal end 1b of the sheath 1.

The treatment unit 110B of the present modified example is configured of the sheath 1 and the inner sheath 21 inserted through the sheath 1. In other words, the treatment unit 110B is configured of a double sheath that extends in the longitudinal direction A.

In the distal end surface 1t of the sheath 1, the suction opening portion 3B is an opening formed between an inner circumferential edge of the suction lumen 30B and an outer circumferential edge of the inner sheath 21.

In the treatment unit 110B, the inner sheath 21 may be provided to be able to project and retract from the suction opening portion 3B.

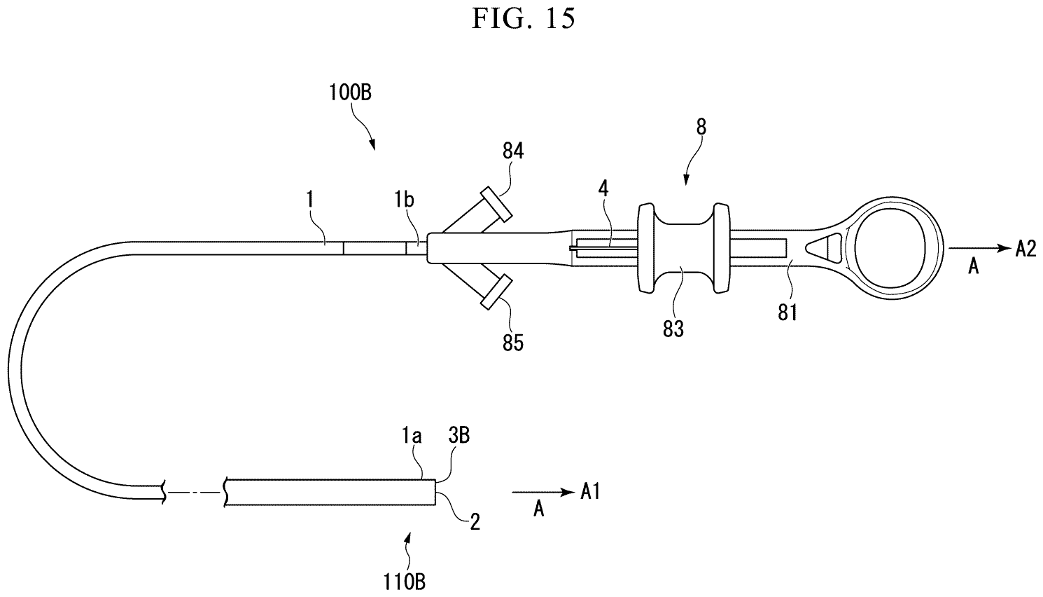

FIG. 15 is an overall view showing an endoscopic device 100B, which is a modified example of the endoscopic device 100.

The endoscopic device 100B includes the sheath 1, an operation wire 4, and the operation unit 8. The above-described treatment unit 110B is provided at a distal end portion of the endoscopic device 100B.

The operation wire 4 is a metal wire, and is connected to a proximal end portion of the inner sheath 21. A proximal end portion of the operation wire 4 is fixed to a slider 83.

The slider 83 is attached to the operation unit main body 81 so that it can move in the longitudinal direction A.

Since the proximal end portion of the operation wire 4 is fixed to the slider 83, the operation wire 4 advances and retracts when the surgeon advances and retracts the slider 83 relative to the operation unit main body 81.

In addition, since the inner sheath 21 is connected to a distal end portion of the operation wire 4, the surgeon can advance and retract the inner sheath 21 by advancing and retracting the operation wire 4, and can cause it to project and retract from the suction opening portion 3B.

### Modified Example 3

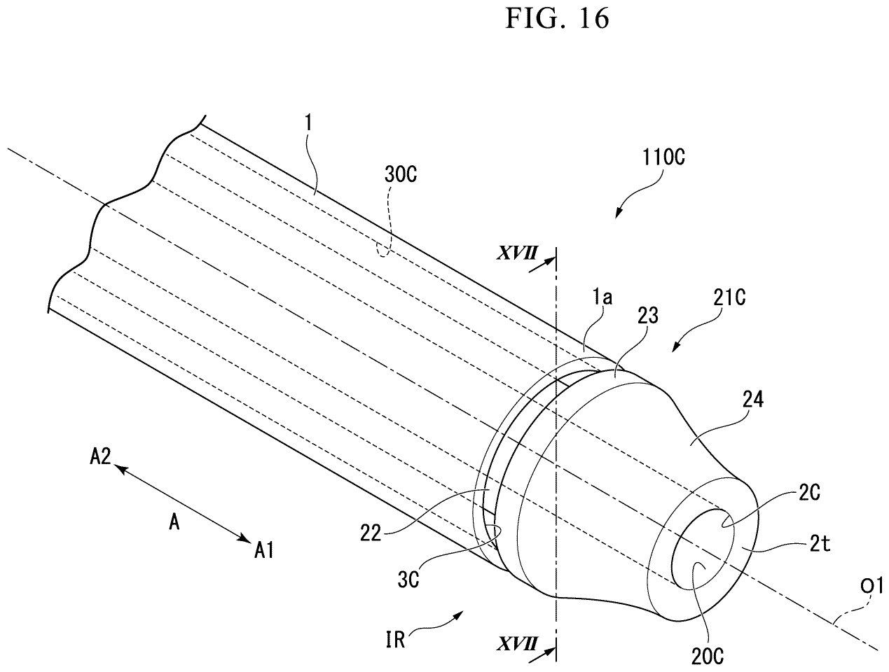

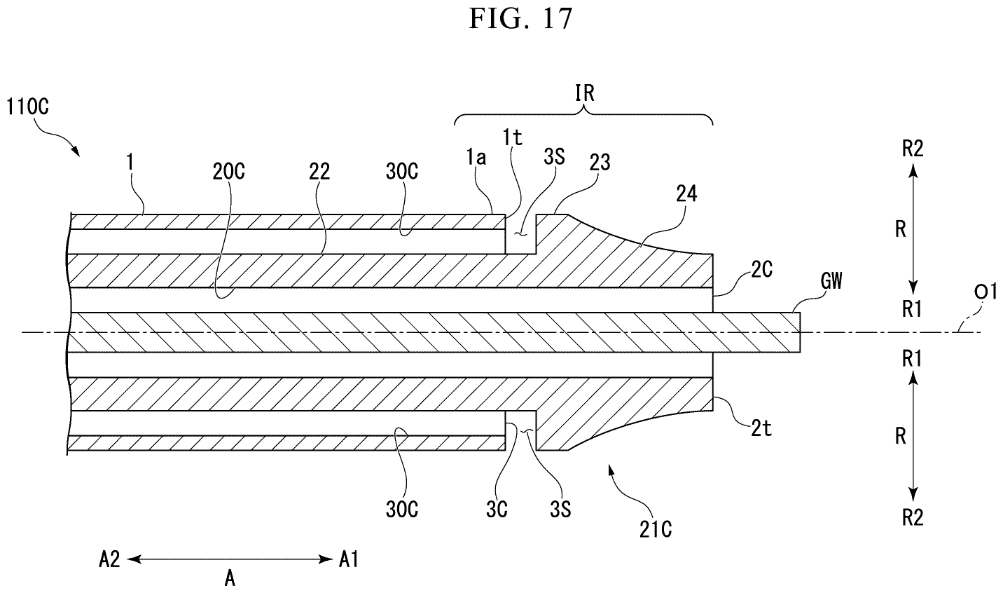

FIG. 16 is a perspective view showing a treatment unit 110C, which is a modified example of the treatment unit 110. FIG. 17 is a cross-sectional view along line XVII-XVII in FIG. 16.

In the treatment unit 110C, a central axis of a suction lumen 30C communicating with a suction opening portion 3C provided on the distal end surface 1t of the sheath 1 coincides with the central axis O1 of the sheath 1. The central axis of the suction lumen 30C does not have to strictly coincide with the central axis O1.

In addition, a treatment tool opening portion 2C is provided in an inner sheath 21C inserted through the suction lumen 30C in the longitudinal direction A.

The inner sheath 21C includes a tube 22 inserted through the suction lumen 30C in the longitudinal direction A, a protruding portion 23 provided at a distal end of the tube 22, and a tapered portion 24 extending from the protruding portion 23 to the distal end side A1.

The treatment tool opening portion 2C, through which the guide wire GW can project and retract, is provided on a distal end surface 2t of the inner sheath 21C. The treatment tool lumen 20C communicating with the treatment tool opening portion 2C is inserted through the inner sheath 21C in the longitudinal direction A.

The tube 22 is an elongated member inserted through the suction lumen 30C. The tube 22 extends, for example, from the distal end 1a to the proximal end 1b of the sheath 1.

The treatment unit 110C of the present modified example is configured of the sheath 1 and the tube 22 inserted through the sheath 1. That is, the treatment unit 110C is configured of a double sheath that extends in the longitudinal direction A.

As shown in FIGS. 16 and 17, the protruding portion 23 is a cylindrical member whose central axis extends in the longitudinal direction A. The protruding portion 23 does not have to be strictly cylindrical.

An outer diameter of the protruding portion 23 is greater than a diameter of the suction opening portion 3C. That is, the protruding portion 23 protrudes toward the outer side R2 of the suction opening portion 3C in the radial direction R of the sheath 1.

The protruding portion 23 is disposed on the distal end side A1 from the suction opening portion 3C. Since the protruding portion 23 has the outer diameter greater than the diameter of the suction opening portion 3C, it is not disposed inside the suction lumen 30C.

The tapered portion 24 is a truncated cone-shaped member whose diameter decreases toward the distal end side Al and whose central axis extends in the longitudinal direction A. The tapered portion 24 does not have to be strictly truncated cone-shaped.

The inner sheath 21C is configured by connecting the tube 22, the protruding portion 23, and the tapered portion 24 in order from the proximal end side A2.

The treatment tool lumen 20C is inserted through the tube 22, the protruding portion 23, and the tapered portion 24 in the longitudinal direction A.

In the distal end surface 1t of the sheath 1, the suction opening portion 3C is an opening formed between an inner circumferential edge of the suction lumen 30C and an outer circumferential surface of the tube 22.

As shown in FIG. 17, the protruding portion 23 covers the suction opening portion 3C from the distal end side A1. In addition, the protruding portion 23 is disposed at a distance from the suction opening portion 3C to the distal end side A1.

In the longitudinal direction A, a gap (suction space 3S) of a predetermined size is formed between the suction opening portion 3C and the protruding portion 23.

The suction space 3S is formed around the entire circumference of the treatment unit 110C in the circumferential direction C. The suction space 3S is a space that communicates with the suction lumen 30C. For that reason, when the suction lumen 30C is under negative pressure, the suction space 3S is also under negative pressure.

As shown in FIG. 17, the treatment tool opening portion 2C, the suction opening portion 3C, and the suction space 3S are provided in the insertion region IR.

When the distal end portion of the sheath 1 (here, the insertion region IR of the treatment unit 110C) is inserted into the bile and pancreatic ducts B in the above-described sheath insertion step and the suction lumen 30C is under negative pressure in the above-described suction step, the suction opening portion 3C sucks the inner wall of the bile and pancreatic ducts B through the suction space 3S.

The inner sheath 21C is provided to be able to advance and retract relative to the sheath 1 by the operation wire 4 and the slider 83, for example, in the same manner as the above-described inner sheath 21.

The surgeon can change a distance in the longitudinal direction A between the protruding portion 23 and the suction opening portion 3C by advancing and retracting the inner sheath 21C relative to the sheath 1.

By changing the distance in the longitudinal direction A between the protruding portion 23 and the suction opening portion 3C, a size of the suction space 3S in the longitudinal direction A formed between the protruding portion 23 and the suction opening portion 3C is changed.

The surgeon can change an area of a suction port that sucks the inner wall of the bile and pancreatic ducts B in the treatment unit 110C by advancing and retracting the inner sheath 21C to change the size of the suction space 3S.

For example, when a suction force sucking the inner wall of the bile and pancreatic ducts B is excessive, there is a possibility that the inner wall of the bile and pancreatic ducts B will be invaded. Invasion to the inner wall of the bile and pancreatic ducts B indicates damage to the living tissue in the inner wall of the bile and pancreatic ducts B.

Also, when the suction force for sucking the inner wall of the bile and pancreatic ducts B is insufficient, there is a possibility that sufficient traction of the papilla PA will not be possible, and the guide wire GW is less likely to be inserted from the opening portion PA1.

The surgeon can change the suction force for sucking the inner wall of the bile and pancreatic ducts B by advancing and retracting the inner sheath 21C to change the area of the suction port in the treatment unit 110C, and can suck the inner wall of the bile and pancreatic ducts B with a suction force that takes into account the degree of invasion of the inner wall of the bile and pancreatic ducts B and an adhesion force due to suction.

Further, the tapered portion 24 of the inner sheath 21C is narrowed toward the distal end side A1, and thus, in the above-described sheath insertion step, the surgeon can easily insert the inner sheath 21C into the inside of the bile and pancreatic ducts B from the opening portion PA1.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscopic device comprising:
a sheath extending in a longitudinal direction thereof;
a treatment tool opening portion that communicates with a treatment tool lumen inserted through the sheath in the longitudinal direction and allows a treatment tool inserted through the treatment tool lumen to project to and retract from a distal end side of the sheath in the longitudinal direction; and
a suction opening portion communicating with a suction lumen inserted through the sheath in the longitudinal direction,
wherein the treatment tool opening portion and the suction opening portion are provided in an insertion region that is disposed inside bile and pancreatic ducts when the sheath is inserted into the bile and pancreatic ducts, and
the suction opening portion is able to suck an inner wall of the bile and pancreatic ducts.

2. The endoscopic device according to claim 1,
wherein the suction opening portion is provided on an outer side of the treatment tool opening portion in a radial direction of the sheath.

3. The endoscopic device according to claim 2,
wherein the suction opening portion is provided on a distal end surface of the sheath.

4. The endoscopic device according to claim 2,
wherein the suction opening portion is provided on an outer circumferential surface of the sheath.

5. The endoscopic device according to any one of claim 1,
wherein a plurality of suction opening portions are provided, and
the plurality of suction opening portions are provided at equal intervals in a circumferential direction of a central axis of the sheath.

6. The endoscopic device according to claim 1, further comprising:
an inner sheath inserted through the suction lumen,
wherein the treatment tool opening portion communicates with the treatment tool lumen inserted through the inner sheath in the longitudinal direction.

7. The endoscopic device according to claim 1, further comprising:
an inner sheath including a tube inserted through the suction lumen and a protruding portion that is provided on the distal end side from the suction opening portion and protrudes toward an outer side in a radial direction of the sheath from the suction opening portion,
wherein the treatment tool opening portion communicates with the treatment tool lumen inserted through the inner sheath in the longitudinal direction, and
the suction opening portion sucks the inner wall of the bile and pancreatic ducts via a suction space formed between the suction opening portion and the protruding portion in the longitudinal direction.

8. A treatment tool insertion method comprising:
a sheath insertion step of inserting a distal end portion of a sheath extending in a longitudinal direction thereof into an inside of bile and pancreatic ducts through an opening portion of a papilla;
a suction step of sucking an inner wall of the bile and pancreatic ducts using a suction opening portion provided in an insertion region of the sheath inserted into the inside of the bile and pancreatic ducts;
a traction step of performing traction of the sheath, which has sucked the inner wall of the bile and pancreatic ducts, toward a proximal end side in the longitudinal direction, thereby causing the sheath to perform traction of the papilla; and
a treatment tool insertion step of advancing a treatment tool through a treatment tool opening portion of the sheath, which has performed traction of the papilla, and inserting the treatment tool into the inside of the bile and pancreatic ducts.

9. The treatment tool insertion method according to claim 8,
wherein, in the suction step, the inner wall of the bile and pancreatic ducts is sucked by the suction opening portion provided on an outer side in a radial direction of the sheath from the treatment tool opening portion.

10. The treatment tool insertion method according to claim 8,
wherein, in the suction step, the inner wall of the bile and pancreatic ducts is sucked by the suction opening portion provided on a distal end surface of the sheath.

11. The treatment tool insertion method according to claim 8,
wherein, in the suction step, the inner wall of the bile and pancreatic ducts is sucked by the suction opening portion provided on an outer circumferential surface of the sheath.

12. The treatment tool insertion method according to any one of claim 8,
wherein, in the suction step, the inner wall of the bile and pancreatic ducts is sucked by a plurality of suction opening portions provided at equal intervals in a circumferential direction of a central axis of the sheath.

13. The treatment tool insertion method according to claim 8,
wherein, in the treatment tool insertion step, the treatment tool is advanced from the treatment tool opening portion provided in an inner sheath inserted through a suction lumen communicating with the suction opening portion.

14. The treatment tool insertion method according to claim 8,
wherein, in the suction step, the inner wall of the bile and pancreatic ducts is sucked via a suction space formed between a protruding portion provided at a distal end portion of an inner sheath inserted through a suction lumen communicating with the suction opening portion and the suction opening portion in the longitudinal direction, and
in the treatment tool insertion step, the treatment tool is advanced from the treatment tool opening portion provided in the inner sheath.
